(19) United States
(12) Patent Application Publication
(10) Pub. No.: US 2015/0182149 A1
(43) Pub. Date: Jul. 2, 2015

(54) METHODS FOR OPTIMALLY MATCHING MUSICAL RHYTHMS TO PHYSICAL AND PHYSIOLOGIC RHYTHMS

(71) Applicant: Simbionics, Boston, MA (US)

(72) Inventors: Benjamin I. RAPOPORT, New York, NY (US); Craig MERMEL, Brighton, MA (US)

(73) Assignee: Simbionics, Boston, MA (US)

(21) Appl. No.: 14/174,633

(22) Filed: Feb. 6, 2014

Related U.S. Application Data
(60) Provisional application No. 61/921,209, filed on Dec. 27, 2013.

(51) Int. Cl. A61B 5/11; G10H 7/00

(52) U.S. Cl. CPC A61B 5/1123; G10H 7/00

(57) ABSTRACT

A set of methods is described for identifying optimal repetition rates for certain repetitive processes, and identifying musical selections with tempi matched to those optimal rates, so as to use synchrony with musical rhythms as a guide to optimizing performance in repetitive physical, biomechanical, and physiologic processes.

### RELATED APPLICATIONS

This application claims priority to pending U.S. Provisional Application No. 61/921,209 filed Dec. 27, 2013 entitled “Methods for Optimally Matching Musical Rhythms to Physical and Physiological Rhythms” the disclosure for which is incorporated in its entirety herein by reference.

This application also includes by reference U.S. patent application Ser. No. 14/145,042 filed Dec. 31, 2013 entitled “Method for Determining Aerobic Capacity” which claims priority to U.S. Provisional Application No. 61/880,528 filed Sep. 20, 2013 entitled “Method for Determining Aerobic Capacity.” This application hereby incorporates all cited references in their entirety.

### BACKGROUND

Repetitive, rhythmic physical activities involve transformation of energy from metabolic to mechanical form. This energy transfer takes place with each cycle or repetition of the stereotyped movement. Each cycle requires an investment of metabolic energy. The output can typically be measured in terms of total body kinetic energy, as in the horizontal translation of a runner or cyclist, in which each stride or stroke of the pedal contributes to maintaining a forward velocity. Thus, in such activities there is a transformation of metabolic to mechanical energy that takes place with a frequency equal to the cadence of the activity, as in the running stride or pedal stroke rate of the runner and cyclist, respectively.

A number of approaches have made for measuring and optimizing the repetition rate, or “cadence,” in rhythmic biomechanical and physiologic processes, including athletic and other physical activities. Previous work in this area may be divided into categories as follows:

1. Measurement of cadence, tempo, or frequency in repetitive processes, including biomechanical processes, such as stride rate of a walker or runner, or pedal rate of a bicyclist.

2. General considerations regarding improvement of athletic or exercise performance on the basis of heart rate or biomechanical cadence (as in walking, running, or bicycling).

3. Measurement of the tempo of existing musical selections.

### Scientific Literature

Those skilled in the art are aware of public-domain information concerning optimization of cadence, particularly in activities such as running and bicycling, as well as optimization of associated parameters such as speed, stride length while running, gear ratio while cycling, and schemes for modifying certain of these parameters when others, or external factors such as terrain, incline, or environmental factors such as temperature or wind speed, change. (McArdle, W. D., et al. Exercise Physiology, Lippincott Williams & Wilkins (2009); Brooks, G., et al., Exercise Physiology: Human Bioenergetics and Its Applications, (2004); Noakes, T., Lore of Running, Human Kinetics(2002); Burke, E. R., High-Tech Cycling, (2003). The relationship between stride length and stride frequency in runners has been studied empirically in some detail (McArdle, W. D., et al. Exercise Physiology, Lippincott Williams & Wilkins (2009)), and observations of elite runners have led to speculations regarding optimal running cadences. Cavanagh, P. R. and K. R. Williams, The Effect of Stride Length Variation on Oxygen Uptake During Distance Running, Medicine and Science in Sports and Exercise, Vol. 14(1), pp. 30-35 (1982). Schemes for selecting gear ratios and cadences while cycling have also been described. Burke, E. R., High-Tech Cycling, (2003). Information on these subjects is transmitted both through published scientific and patent literature, and also informally through sharing of customary and best practices among experienced practitioners, including professional athletes and their supporters and advisers.

U.S. Pat. No. 7,841,967: “Method and apparatus for providing fitness coaching using a mobile device” suggests that it provides “A method and apparatus providing fitness coaching including a mobile device to enable a user to receive a tailored fitness session.” However, the system described in that disclosure functions strictly by monitoring heart rate. In particular, it uses heart rate as a measure of aerobic intensity level, and provides recommendations on the basis of this measure.

U.S. Patent No. 7,771,320: “Athletic performance sensing and/or tracking systems and methods” also relates to sensing and recommendations regarding athletic performance. It describes methods and systems for detecting the effort level of an individual engaged in physical exercise based on subjective and objective measurements, and providing audio or visual media content to accompany exercise. It does not disclose optimization methods such as those described herein.

### SUMMARY

There are further key differences between work described in the art and the present disclosure. In particular, the systems and methods described in the prior art do not use or anticipate the precise, personalized, or model-based computations and methodology described in the present disclosure to optimize cadence with respect to fat metabolism, endurance performance, or any of the other parameters or states described here. Furthermore, methods and systems described in the prior art relating to cadence typically describe systems for cadence measurement, or provide general considerations with respect to optimizing cadence during physical activities, rather than systematic, personalized, or model-based cadence optimization. Other methods and systems in the prior art involve matching a music selection to a specified tempo, but do not describe selecting the desired tempo or cadence on the basis of optimality with respect to endurance performance, metabolic states, or other criteria.

In some embodiments, the processes of interest include those of consciously controlled repetitive motor activities such as step rate while walking, running, or dancing. In other embodiments, the physiologic processes of interest include those of autonomically regulated, quasiperiodic physiologic processes such as heart and respiratory rate.

In some embodiments, optimality is defined on the basis of an individual achieving a designated metabolic state during rhythmic physical exercise; music having the appropriate tempo can be played for the user as an entrainment signal, to assist the user in moving with the repetition rate identified as optimal. In other embodiments, optimality is defined on the basis of the actual repetition rate of a process occurring in real time, such as the stride rate or heart rate of a runner or dancer; music having a tempo matched to the observed repetition rate can then be played, automatically synchronizing the musical tempo to the rhythm of the observed process.

The present disclosure provides methods and systems to determine the optimum cadences for rhythmic physical and physiologic processes, defined with respect to particular objectives, and to select and play music with tempo optimally matched to the rhythms of specific physical and physiologic processes. This disclosure introduces a system for empirically determining functional relationships among the tempo (or cadence) of designated physical or physiologic processes, on one hand, and metabolic output during those processes, on the other. Once such relationships have been determined a user may be given auditory guidance, through the tempo of music played, as to the appropriate cadence to adopt during exercise in order to achieve particular metabolic goals (including, but not limited to, maximizing running or cycling speed for a given metabolic power output, or maximizing the rate of fat metabolism). Alternatively, the systems and methods disclosed here can be used to detect the tempo of natural activities (including, but not limited to, walking, running, or cycling) and to select and play music with matching tempo.

A set of methods is described for selecting music with cadence optimally matched to designated physical and physiologic rhythms. These methods function adaptively, automatically, and in real time. The physical and physiologic rhythms of interest include both those of consciously controlled repetitive motor activities such as stride rate while walking or running, as well as those of autonomically regulated, quasiperiodic physiologic processes such as heart rate and respiratory rate. The methods described in this disclosure fall into two classes, respectively designated “Following” and “Leading” techniques. In the “Following” class, a sensor or set of sensors is used to detect a physical or physiologic rhythm, such as heart rate or stride rate, and music with the corresponding cadence is retrieved from a database to match the detected rhythm. In a reversal of the traditional paradigm “Dance to the music,” the “Following” methods are designed to facilitate matching musical rhythms to repetitive physical movements, while also broadening the notion of what constitutes “dance” to any repetitive physical movement or physiologic process. The “Leading” methods, by contrast, are designed to use musical cadence as an entrainment signal to assist individuals in optimizing their metabolic output while engaged in repetitive physical activities. These methods entail first establishing an empirical, functional relationship between physical or physiologic cadence and metabolic output (such as a relationship between heart rate, running stride rate, or bicycling cadence, on the one hand, and total body oxygen uptake during exercise, on the other); then identifying a desired metabolic state (as defined, for example, by a particular level of aerobic intensity with respect to maximum oxygen uptake, and optimized, for example, for time-efficient fat metabolism, or for maximum sustainable running, walking, or cycling speed); and finally identifying the physical cadence that elicits the chosen metabolic state. A selection of music having the same cadence can then be played for the user as an entrainment signal, to assist the user in operating at the cadence identified as optimal.

In one aspect, the present disclosure relates to the measurement and estimation of metabolic and biomechanical power output associated with performing repetitive physical actions, including various athletic activities. It describes a system and set of methods for establishing both theoretically-based and empirically-derived relationships among the cadence or tempo of a physical activity and the metabolic costs associated with performing that activity, and identifying tempos associated with specific values of metabolic costs that optimize specific user-defined objectives. User-defined objectives may include operating at a cadence that maximizes endurance at a given speed, that is associated with the highest rate of fat metabolism for a given level of metabolic power output, or that maximizes speed for a given level of metabolic power output.

In another aspect, the present disclosure relates to the use of music as an entrainment signal for the optimization of specific metabolic and biomechanical states during repetitive physical movements. It describes a system in which the physical rhythm that is optimal for achieving any of a number of specific goal states can be identified and matched to the rhythms present in a database of musical selections, and in which that music can be played back in its original or in a modified form in order to assist the user in achieving the optimum physical cadence.

In another aspect, the present disclosure relates to the use of music to ‘follow’ natural physical rhythms, reversing the traditional paradigm in which physical rhythms ‘follow’ the music, in order to emphasize the dance-like nature of repetitive physical activities. In this mode, the system senses the pace or rhythm of a physical or physiologic process, and dynamically selects and plays music matched to the rhythm of the activity being performed. This mode is a natural extension of the cadence-optimization framework, in which the optimum cadence is defined as the naturally-preferred cadence of a user at each point in time.

In one aspect, the present disclosure relates to a method for determining an optimal repetition rate for a repetitive biomechanical or physiologic process of a user. In some embodiments, the method can include defining an optimization objective based on a metabolic cost function; storing the optimization objective in memory; monitoring, using a sensor, a repetition rate of the biomechanical or physiologic process; estimating a functional dependence of expended metabolic energy on the repetition rate, based on the optimization objective; and identifying an optimum repetition rate for the repetitive biomechanical or physiological process based on the functional dependence. In some embodiments, the metabolic cost function can include maximizing or minimizing expended energy during the repetitive biomechanical or physiologic process. In some embodiments, the metabolic cost function can include maximizing or minimizing fat or carbohydrate metabolism during the repetitive biomechanical or physiologic process. In some embodiments, the method can include constraining the user to walking, running, cycling, dancing, or performing a form of repetitive manual labor within a range of speeds. In some embodiments, the metabolic cost function identifies the repetition rate associated with the maximum speed at which a user is able to walk, run, cycle, dance, or perform a form of repetitive manual labor. In some embodiments, the method can include constraining the user to cover a particular distance, or constraining the total energy to be expended by the user over a period of time. In some embodiments, the biomechanical or physiologic process can include a consciously regulated process. In some embodiments, the consciously regulated process can include one of walking, running, cycling, dancing, or performing a form of repetitive manual labor. In some embodiments, the biomechanical or physiologic process can include an unconsciously or autonomically regulated process. In some embodiments, the unconsciously or autonomically regulated process can include one of heart rate or respiratory rate. In some embodiments, the biomechanical or physiologic process comprises a quasiperiodic process.

In some embodiment, the user can include an animal, for example, one of a human, a horse, or a dog. In some embodiments, the method can include selecting music matched to the optimal repetition rate. In some embodiments, the matching of the music to the optimal repetition rate is based on tempo. In some embodiments, the matching of the music to the optimal repetition rate is based on an observed relationship between specific features of the music and a user's past repetition rate while listening to the music. In some embodiments, the matching of the music to the optimal repetition rate is based on the observed relationship between specific features of the music and the repetition rates observed in a population of users in performing a repetitive process while listening to said music or music with similar features. In some embodiments, the specific features of the music can be tempo, key, musical genre, artist, or mood. In some embodiments, the method can include playing the selected music for the user to provide synchrony with musical rhythms as a guide to optimizing performance in the repetitive biomechanical or physiologic process.

In some embodiments, the method can include distorting the musical selection based on a function of the degree to which the observed repetition rate differs from the rate identified as optimal in order to guide a user in modifying actions to operate at the optimal repetition rate. In some embodiments, the optimization objective is defined on the basis of an individual achieving a designated metabolic state during rhythmic physical exercise, and wherein music having the appropriate tempo is played for the user as an entrainment signal, to assist the user in moving with an identified optimal repetition rate.

In one aspect, the present disclosure relates to a method of optimally matching musical rhythms to physical, biomechanical, and physiologic rhythms, wherein optimality is defined on the basis of an actual repetition rate of a process occurring in real time. In some embodiments, the method can include continually sensing, using a sensor, a repetition rate of a physical, biomechanical, or physiologic process; automatically selecting music matched by tempo to the repetition rate of the process of interest; and playing the selected music for the user, as the user moves with the detected repetition rate.

In some embodiments, the method can include adjusting the selected music as the repetition rate varies over time. In some embodiments, the biomechanical or physiologic process can include a consciously regulated process. In some embodiments, the biomechanical or physiologic process can include one of walking, running, cycling, dancing, or performing a form of repetitive manual labor. In some embodiments, the biomechanical or physiologic process can include one of an unconsciously or autonomically regulated process. In some embodiments, the unconsciously or autonomically regulated process can include one of heart rate or respiratory rate. In some embodiments, the physical process can include a repetitive process, identified as desirable by a user for the purposes of guiding or automatically matching musical tempo. In some embodiments, the biomechanical or physiologic process can include a quasiperiodic process. In some embodiments, the user can be an animal, for example, a human, a horse, or a dog. In some embodiments, the method can include aggregating data on a difference between optimal and observed repetition rate for a given musical selection. In some embodiments, the statistical properties of the aggregated data are used to define a metric In some embodiments, the data are aggregated over a multiplicity of users. In some embodiments, the metric describes the degree to which the observed repetition rate matches the optimal repetition rate while the music is being played. In some embodiments, the metric is used to guide the music selection.

### BRIEF DESCRIPTION OF THE DRAWINGS

FIG. 1 is a block diagram overview of a system for optimally matching musical rhythms to physical and physiologic rhythms of an individual user, according to embodiments of the present disclosure.

FIG. 2 is a flowchart illustrating a method for optimally matching musical rhythms to physical and physiologic rhythms of an individual user, utilized by the system shown in FIG. 1, according to embodiments of the present disclosure.

FIG. 3 is a flowchart illustrating a sub-method, for acquiring and storing biometric and anthropometric data from a user, utilized within the system for optimally matching musical rhythms to physical and physiologic rhythms of an individual user (shown in FIG. 1) according to embodiments of the present disclosure.

FIG. 4 is a flowchart illustrating sub-methods, for acquiring and storing data from sensors monitoring physiologic, biomechanical, geophysical, or other parameters associated with a user, utilized within the system for optimally matching musical rhythms to physical and physiologic rhythms of an individual user (shown in FIG. 1) according to embodiments of the present disclosure.

FIG. 5 is a flowchart illustrating a sub-method, for calibrating a subsystem designed to match musical rhythms optimally to physical and physiologic rhythms of an individual user (shown in FIG. 1) according to embodiments of the present disclosure.

FIG. 6 is a flowchart illustrating a sub-method in the system designed to match musical rhythms optimally to physical and physiologic rhythms of an individual user (shown in FIG. 1), according to embodiments of the present disclosure.

FIG. 7 is a flowchart illustrating a sub-method, for identifying a musical selection on the basis of a selected “tempo” (herein the term “cadence” is often used interchangeably with “tempo”), utilized within the system for matching musical rhythms optimally to physical and physiologic rhythms of an individual user (shown in FIG. 1), according to embodiments of the present disclosure.

FIG. 8 illustrates the essential features of the Estimation Routine to Compute Optimum Cadence from Sensor Data, according to embodiments of the present disclosure.

FIG. 9 diagrams the operation of the system in Feedforward (“Leading”) mode, according to embodiments of the present disclosure.

FIG. 10 diagrams the operation of the system in Feedback (“Following”) mode, according to embodiments of the present disclosure.

### DESCRIPTION

The present disclosure is directed to systems and methods for determining the optimum repetition rates (herein referred to as “cadences”) for rhythmic physical and physiologic processes, defined with respect to particular objectives, and to identifying and playing music matched in tempo to these optimum cadences.

The need for explicit instruction or supervision when learning and refining complex motor behaviors is a familiar experience; dancing is but one example of a rhythmic activity that practitioners cannot typically perfect without input from an instructor or experienced observer. Experimental work in neuromuscular physiology has demonstrated that the human neuromuscular system is in many cases not capable of optimizing even simple, stereotyped movements through independent learning, but that individuals can learn to optimize such movements when taught the explicit techniques they cannot discover independently through iteration, trial-and-error, or gradient-descent learning Scheidt, R. A. et al, (2011), “Patterns of hypermetria and terminal cocontraction during point-to-point movements demonstrate independent action of trajectory and postural controllers”, Journal of Neurophysiology 106(5): 2368-2382. These findings apply to running, walking, cycling, and other athletic activities in a number of ways, including that runners, walkers, cyclists, and other athletes may not intuit the optimum cadences for their particular objectives for speed, endurance, or metabolic output. (Cavanagh, P. R. and K. R. Williams, The Effect of Stride Length Variation on Oxygen Uptake During Distance Running, Medicine and Science in Sports and Exercise, Vol. 14(1), pp. 30-35 (1982).) In colloquial terms, movement that “feels right” may not always be optimal according to certain objective measures, and approaching optimality may require explicit guidance from an external source.

The relationships among relative and absolute aerobic intensity, overall metabolic rate, rates of fat and carbohydrate metabolism, heart rate, oxygen uptake (VO₂) and maximal oxygen uptake (VO₂max), as well as some additional parameters have been characterized in the scientific literature. (Rapoport, B. I. “Metabolic Factors Limiting Performance in Marathon Runners”, Public Library of Science Computational Biology, Vol. 6(10) (2010) e1000960.) Once a reliable mapping between heart rate and relative aerobic intensity has been established for a given individual, it is possible to predict total metabolic rate as well as fat and carbohydrate metabolism on the basis of observed heart rate. Monitoring and controlling heart rate during exercise is therefore approximately equivalent to monitoring and controlling certain useful aspects of the overall metabolic state of an individual. One aspect of the present disclosure therefore concerns determining heart rates for an individual that are associated with desired metabolic states, identifying musical selections with tempi matched to the appropriate heart rates, and playing those musical selections for a user as auditory cue signals designed to assist the user in synchronizing his or her heart rate to a rate associated with a desired “optimal” overall metabolic state, such as the aerobic intensity at which a user metabolizes the greatest amount of fat in the least amount of time.

Several aspects of the present disclosure relate to identifying an optimal repetition rate, herein and elsewhere in the art referred to as “cadence,” for fundamental component movements in athletic activities characterized by rhythmic, repetitive physical actions, including (but not limited to) running, walking, dancing, and cycling.

Certain engineering principles applicable to the optimization of such systems involving cyclic energy transfer will be familiar to those skilled in the art. The present disclosure describes a phenomenon that is herein termed “physiologic impedance matching,” and refers the optimization of energy transfer efficiency between physiologic processes. The notion of impedance matching in several domains of engineering refers to the design of a coupling between systems, one of which transfers energy to the other, such that the efficiency of the energy transfer is maximized. In particular, when a power source is configured to drive a load, matching the output impedance of the power source to the input impedance of the load maximizes the transfer of power from source to load, and minimizes power lost in the transfer or reflected from the load back to the power source. Impedance matching is a well-known design practice in electrical, mechanical, hydraulic, optical, acoustic, telecommunication, and other domains of engineering. The role of impedance matching in biologic and physiologic processes has been less well explored. The present disclosure is concerned primarily with impedance matching in the energy transfer processes between active muscles performing metabolic work (manifest in the form of chemical reactions in muscle tissue and in the mechanical contraction of muscle fibers), and the power delivered to a body at the level of large-scale movements, including limb movement and full-body translation, as occurs, for example, in many types of athletic activity (including but certainly not limited to running, walking, dancing, bicycling, swimming, and sports such as soccer, basketball, tennis, baseball, and many others). The physiology and mathematical modeling underlying the concept of physiologic impedance matching have not yet been completely characterized, but the present disclosure develops these subjects to an extent sufficient to construct novel, nonobvious, and useful devices for optimizing human performance.

A familiar application of physiologic impedance matching is found in the design of bicycles, which are typically configured with sets of gears that permit the cyclist to vary the number of revolutions of the pedals associated with a full revolution of the bicycle wheels; equivalently, the function of bicycle gears is to enable the cyclist to vary the pedal rate associated with a particular translational speed. Optimizing energy transfer between the metabolic and muscular work performed by the cyclist, on one hand, and the forward movement of the bicycle, on the other, requires selecting settings for the coupling mechanism. In the case of a bicyclist, this coupling between metabolic work and forward movement is specified by the gear ratio of the bicycle and the pedaling frequency (cadence) used by the cyclist. Similar analogies can be made in running, walking, and other activities. The metabolic energy required to generate a single stride or pedal stroke varies (on level terrain) as a function of stride length for a runner, and as a function of gear ratio for a cyclist; the metabolic power (time rate of energy expended) is therefore a function of stride length and frequency in running, and of gear ratio and pedaling cadence for a cyclist. Given a particular gear setting or stride length, the pedal cadence or stride cadence determines a transformation between metabolic power input and mechanical power output.

The functional relationships among biomechanical cadence and such parameters as metabolic power output; oxygen uptake; heart rate; and speed in running, cycling, or other activities, can be determined empirically and in a natural environment, as the present disclosure describes. The general approach to characterizing such functional relationships involves measuring and recording biomechanical cadence together with each of the covarying parameters of interest, and observing their natural or induced covariation over time as a particular subject engages in the activity of interest.

The present system is made possible in part by the widespread availability of portable devices with embedded biophysical and geophysical sensors, including but not limited to accelerometers, gyroscopes, heart rate monitors, and global positioning system transponders. Importantly, the cadence of an individual engaged in repetitive motions such as walking or running can easily be determined by counting relative peaks in the components of acceleration data (often using only the vertical component will suffice), one method among several that underlie the function of existing pedometers, and which are well known to those skilled in the art. In addition, the present authors recently described methods for monitoring metabolic parameters of interest, including maximum oxygen uptake, carbohydrate and fat utilization rates, and lactate production, in U.S. Patent Application No. 61/880,528, which is incorporated by reference in this application.

We describe methods in which the natural variation in physiologic processes and human activities can be observed and analyzed to characterize these relationships, without necessarily requiring users to engage in explicit protocols designed to explore available parametric spaces. These methods enable the systems and methods described here to function with minimal interference in the natural activities of a user.

Once functional relationships between cadence and other parameters of interest have been characterized empirically for a particular individual, it is possible to identify specific cadences associated with particular states, including (to use examples applicable to athletics and exercise) cadences associated with metabolic and performance optima, such as maximal biomechanical efficiency, maximal total metabolic output (globally or constrained to particular speeds or other parameters), and maximal rate of fat metabolism.

### Music as a Guide to Cadence

Musical rhythms and other auditory signals have been used since antiquity to guide, synchronize, and regulate the pace of human performance in repetitive activities. This disclosure describes methods of determining musical tempi matched to specific repetition frequencies of physiological, biomechanical, or other processes. In particular, the present disclosure describes systems and methods, both analytic and empirical, for determining optimal cadences for such processes, and then using the determined optima to guide selection of music with appropriately matched tempi. The musical selections made using these methods can then be used to provide rhythmic auditory cues to guide and optimize performance with respect to cadence.

### Cadence as a Guide to Music

The system and methods disclosed here also permit modes of operation in which the cadence of an activity (such as the stride rate of a person walking, or the repetition frequency of a person engaged in a repetitive task) or processes (such as heart rate) is sensed, and music is selected to match the sensed cadence. In these modes of operation the activity or process whose cadence is sensed may be selected and changed, and variations in cadence may also be identified, so as to make corresponding tempo changes in musical selections. However, the aim in these modes of operation is not necessarily to induce an optimal cadence, but rather to provide music with a tempo that matches the cadence of a process or activity whose repetition frequency is determined by other factors.

As an intuitive example of the counterintuitive usefulness of such modes of operation, consider dancing as an area of application. Typically, dancing requires dancers to match their steps to the beat a piece of music. By contrast, the modes of operation described here will permit a dancer to dance at his or her desired tempo, and then after sensing and analyzing the movements of the dancer, will provide music with a matching tempo. In colloquial terms, in these modes of operation the notion that “The dancer moves with the music” is reversed, and “The music moves with the dancer.”

### Applications

Many potential applications of quantitative, personalized techniques for identifying optimal cadences are not adequately addressed by state-of-the-art methods. Notable examples include:

A. The ability to identify specific physical cadences correlated with desired physiologic states. For example, specific stride rates of walkers and runners, and pedaling rates in bicyclists, are associated with optimal biomechanical efficiency (lowest metabolic power output for a given speed). Additionally, at any specified speed, specific stride rates and pedaling rates are associated with particular levels of aerobic intensity, including intensity levels of specific interest such as the level that maximizes the rate of fat metabolism. Cadence optimization can therefore be used as a means of achieving such desired metabolic states.

B. The ability to dynamically select and play music with tempo matching the cadence of physical or physiologic phenomena in a particular user. For example, music can be selected and played to match the heart rate or respiratory rate of a particular individual, to match the stride rate of a walker or runner, or to match the step rate of a dancer (reversing the traditional paradigm in which a dancer synchronizes his or her movements to the music, the system described herein can dynamically select and play music synchronized to the movements of a dancer).

To address shortcomings in the prior art, the present disclosure introduces a new system and set of methods for optimizing cadence. There are several principal advantages to the system and methods described here:

1. Optimal cadences identified in the scientific literature with respect to certain activities and objectives are not always clearly generalizable to users with characteristics different from the subjects studied. The methods and systems of the present disclosure may be applied to any specific individual to determine personalized optima.

2. The scientific literature does not provide data on the metabolic cost or biomechanical efficiency of all conceivable rhythmic physical and physiologic activities. Using the methods and systems of the present disclosure, a user may compute optimal cadences with respect to personally defined criteria not addressed by the scientific literature.

3. Individually defined optima may change over time as the physiology of an individual changes. The methods and systems of the present disclosure may be applied repeatedly over time, with minimal material or temporal cost to the user, to recompute personalized optima.

4. The entertainment value or other merits of dynamically providing music synchronized in tempo to the rate of periodic and quasiperiodic physiologic, biomechanical, or other processes has not been thoroughly explored in the scientific literature or by systems available in the public domain. The methods and systems of the present disclosure facilitate such exploration.

Exploring each of these advantages in turn, consider the case in which an individual wishes to engage in a walking program for fitness, designed to promote the greatest amount of fat loss under the constraints of not being able to tolerate walking speeds faster than four miles per hour, and being able to devote no more than thirty minutes per day to exercise. The system described herein would begin by passively monitoring aerobic power output while the individual walked, automatically sensing natural variations in walking cadence, and constructing a functional relationship among aerobic power output, walking speed, and stride cadence. As described in the scientific literature (Rapoport, B. I. Metabolic Factors Limiting Performance in Marathon Runners, Public Library of Science Computational Biology, Vol. 6(10) (2010) e1000960), particular levels of aerobic intensity are associated with maximal fat metabolism; the system would proceed to identify the stride cadence at which fat metabolism was achieved while walking at four miles per hour. It would then play music at the associated tempo to promote walking at the optimal stride rate.

Next, consider the case of a runner wishing to maximize her speed over a given distance. Her aerobic capacity limits her to generating at most a specific maximum aerobic power over a given time interval. Using an analytic approach similar to the one described for the walker of the preceding paragraph, the system described herein can establish a functional relationship among cadence, speed, and aerobic power output. Such functional relationships have been described in the scientific literature, but the ability to estimate them for given individuals repeatedly in natural settings, using naturally occurring observed variation rather than specified exercise protocols, has not been described. Running stride rate (cadence) associated with minimal power output (maximal biomechanical efficiency) can then be identified at any chosen speed, enabling the runner to maximize her endurance at top speed (or any speed). The system can also play music at the designated tempo, as a mechanism of entraining the runner to her optimal stride rates. Of note, by providing personally optimized cadences, these methods would free the runner of dependence on “conventional wisdom” and published average optimum stride rates, which may not be personally applicable to her.

Next, consider the case of an individual engaged in a rhythmic activity not typically studied in the scientific literature, such as a repetitive form of manual labor. The methods described for the walker and the runner apply similarly to such an individual, aiding in the identification of work rhythms that promote biomechanical efficiency and endurance, and in the provision of musical accompaniment to such activities.

Next, suppose all of the individuals described in the preceding three paragraphs wish to recompute their optima at one-year, one-month, one-week, or even one-day intervals. The system and methods described herein facilitate continued reevaluation over time, by aggregating user data over time and continually refining estimates of optima. The user need not specify a desire to recompute or reevaluate; the system continually acquires data, using inherent statistical variation in natural activities as a form of natural experimental protocol. Refined optimization estimates are continually made available to the user.

Finally, consider the example of an individual who enjoys dancing but has trouble synchronizing his steps to the music. Such an individual may prespecify a list of musical selections he enjoys, and may proceed to dance; the system will detect his steps and natural cadence, even as the cadence may change, and dynamically identify and play music from the designated library of preferred selections, matching musical tempo to the steps of the dancer.

Several of the sub-methods described herein are similar or identical to those described in U.S. Patent Application No. 61/880,528, which is included by reference in this application.

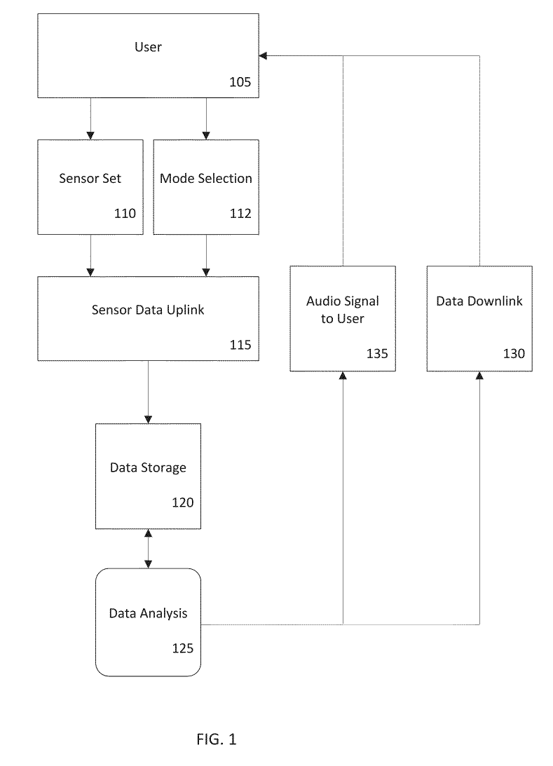

Turning to the drawings, FIG. 1 provides an overview of the system architecture. The system includes a number of sensors 110 that collect information about each user 105 of the system. The sensors 110 most importantly include heart rate monitors, global positioning system (GPS) transponders, and accelerometers. The system is in principle compatible with any type of wearable sensor that tracks these parameters (use of other types of sensor is envisioned as well).

Prior to beginning an activity, each user selects an operation mode 112 that determines whether the user would like the system to operate in ‘Calibration’, ‘Following’ or ‘Leading’ mode, as described in detail in the text that follows. The sensors 110 in turn transmit the information they collect from each user 105 during an activity to a data storage subsystem 120 through a sensor data uplink 115.

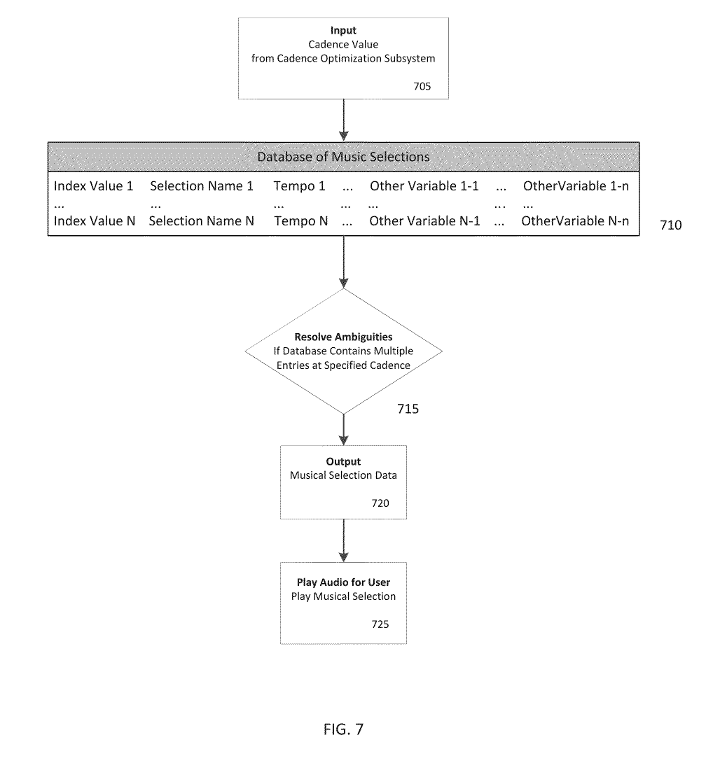

A data analysis subsystem 125 has continuous access to the data accumulated in the data storage subsystem 120, and continuously performs computations as diagrammed in subsequent figures and as described in further detail herein, using data obtained from the sensors mentioned in the previous paragraphs to estimate physical or physiologic cadence and metabolic power output by the individual user wearing the sensors. The results of these computations may be stored in the data analysis subsystem 125, and may also be transmitted to the original user 105 through a data downlink 130. Most importantly, the data analysis subsystem 125 performs a comparison between the current physical cadence and the cadence that is deemed optimal with respect to the mode 112 that has been chosen by the user 105. Using a sub-method described in detail in FIG. 7, the system will send an audio signal 135 back to the user (other forms of feedback signal are envisioned as well, including visual and tactile signals) 105 to assist the user in achieving his or her goal.

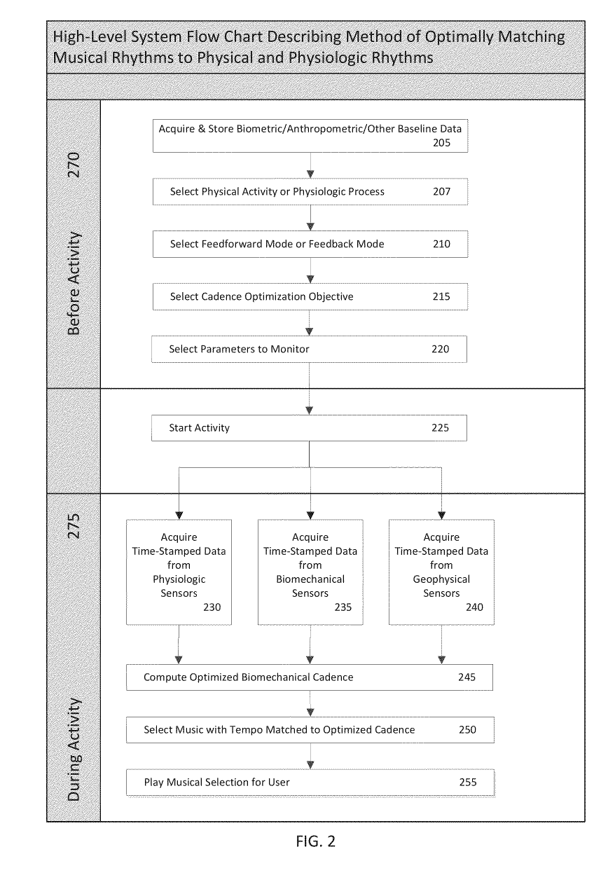

FIG. 2 provides an overview description of the process by which the system can optimally match musical rhythms to physical and physiological cadences. Individual components of this process are diagrammed in subsequent figures and described in further detail herein.

The first phase of the process of matching musical and physical of physiologic cadence begins before any activity commences. This phase is designated “Before Activity” 270, and begins with the “Acquisition and Storage of Biometric/Anthropometric/Other Baseline Data” d205 from each user. Baseline biometric data include weight, age, gender, and height, among other parameters. Anthropometric data include percentage body fat and lean body mass. Other baseline data include, but are not limited to, environmental factors including temperature, humidity, precipitation, barometric pressure, and terrain type (paved road versus trail). The type of physical activity or physiological process that is to be optimized is then selected by the user 207. Types of physical activity supported by this system include essentially any activity in which a physical motion is repeated periodically or quasiperiodically, and include, but are not limited to, running, walking, dancing, bicycling, swimming, and sports such as soccer, basketball, tennis, baseball, and many others. Types of physiological processes supported by this system include but are not limited to those that are under rhythmic autonomic control such as heart rate, blood pressure, and respiratory rate, among others.

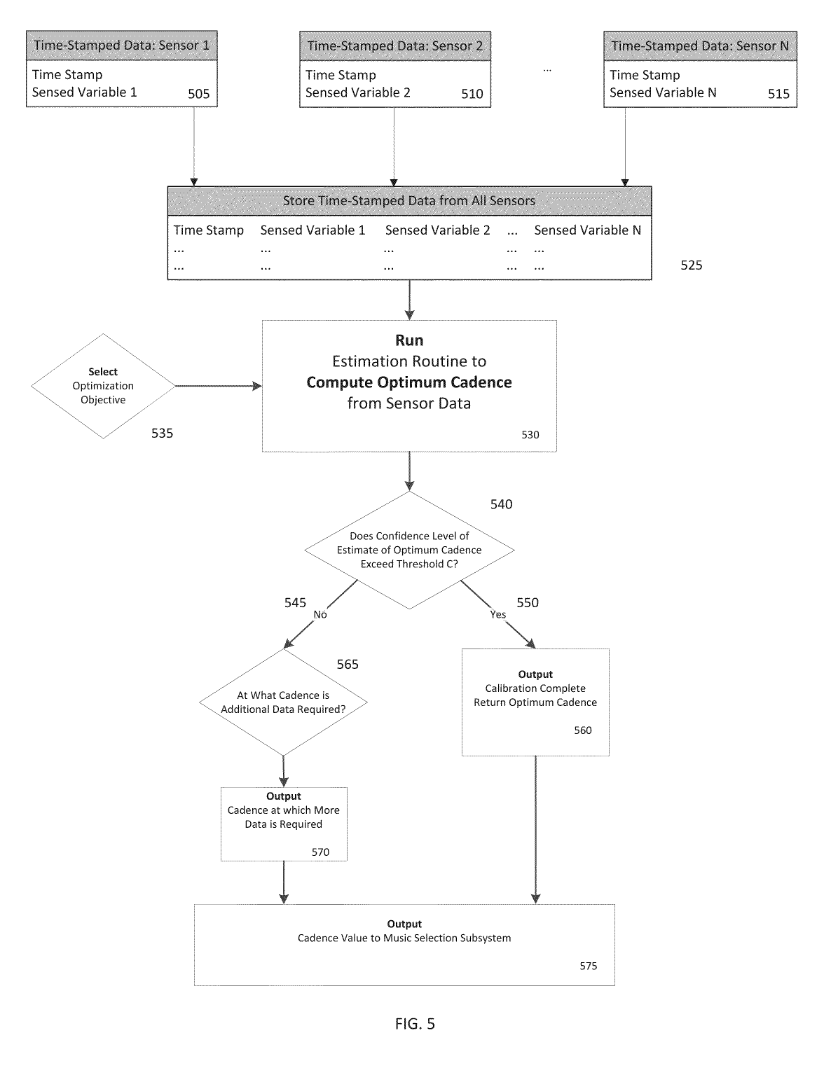

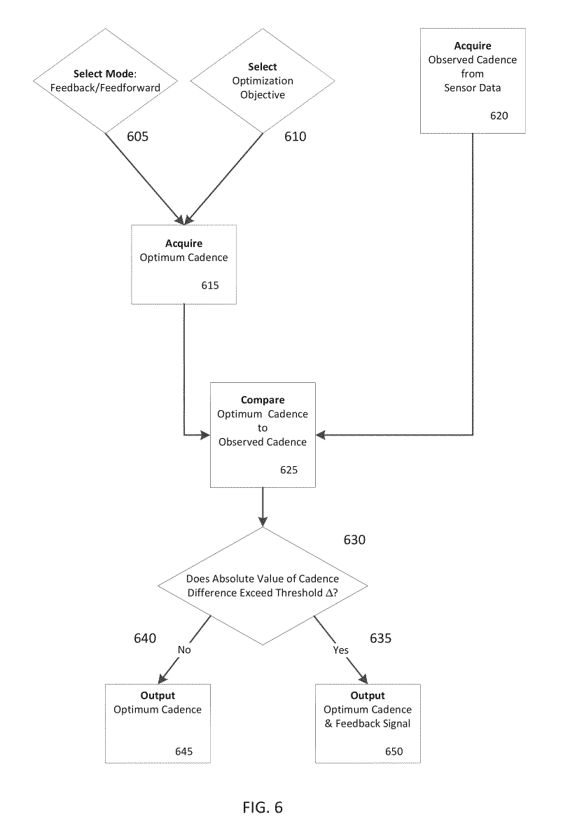

The user then selects the mode of operation of the system 210. Two principal modes of operation are envisioned: Feedforward (“Leading”) and Feedback (“Following”) mode. The feedforward mode requires an initial Calibration phase, which can be considered a third mode. Each of these modes of operation are described in greater detail in FIG. 5 and FIG. 6 as well as in the text herein.

The user next selects an optimization objective 215. In Feedback mode, the optimization objective is automatically selected and is always for the system to generate musical selections that best match the cadence of the physical or physiological process of interest. However, specific details of how the matching process should operate may be chosen by the user at this point, including but not limited to the sensitivity of the system to small changes in cadence and the mechanism for disambiguating among musical selections of identical tempo. In Feedforward mode, the optimization objective may be one of many possible objectives, including but not limited to operating at a cadence that maximizes endurance at a given speed, or that is associated with the highest rate of fat metabolism for a given level of metabolic power output, or that maximizes speed for a given level of metabolic power output.

Based on the physical activity or physiologic process to be monitored 207, the mode of operation 210, and the optimization objective 215, the system will determine which parameters need to be monitored 220. The user will be prompted with instructions to connect the appropriate sensors to the system before the activity may be started 225.

During the activity 275, time series data is acquired from multiple sensors 230, 235, and 240, and stored in appropriate storage subsystems 120. The system then examines the acquired data and estimates an optimum cadence 245 given the optimization objectives. The optimum cadence determined is output to a musical selection subsystem 250, which selects music with tempo matched to the designated optimum cadence. The selected music is then played for the user.

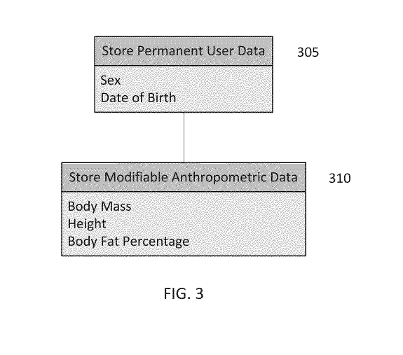

FIG. 3 describes in detail the process labeled “Acquire & Store Biometric/Anthropometric Data” 205 in FIG. 2. In this process, several types of data are acquired from the user.

One type of data, “Permanent User Data” 305, includes variables such as sex and date of birth. Other variables may also be included in this class. The values of these variables are transmitted via the data uplink 115 and are stored in a database of “Permanent User Data” within the main data storage subsystem 120.

A second type of data, “Modifiable Anthropometric Data” 310, includes variables such as body mass, height, and body fat percentage. Other variables may also be included in this class. The values of these variables are transmitted via the data uplink 115 and are stored in a database of “Modifiable Anthropometric Data” within the main data storage subsystem 120.

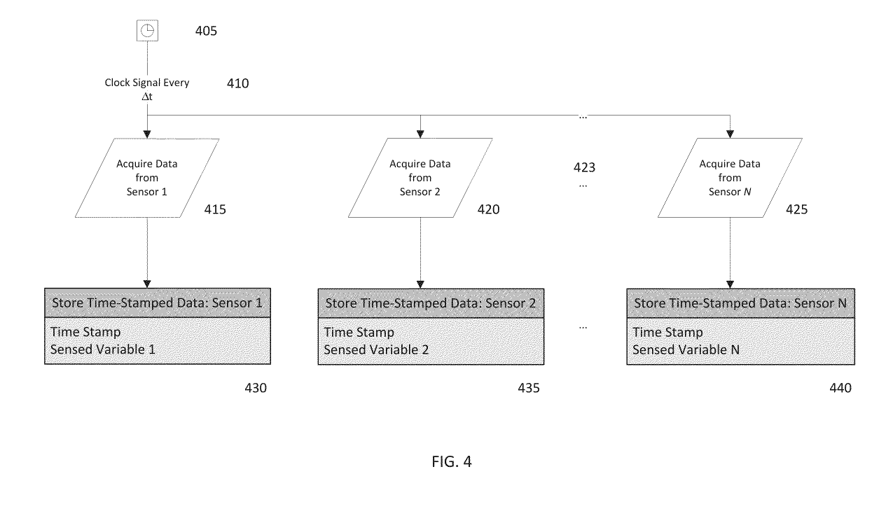

FIG. 4 describes in detail the generic process of acquiring time-stamped sensor data from multiple sensors, which can be used in parallel with appropriate sensors to implement the individual subsystems “Acquire Time-Stamped Data from Physiologic Sensors” 230, “Acquire Time-Stamped Data From Biomechanical Sensors” 235, and “Acquire Time-Stamped Data from Geophysical Sensors” 240, as diagrammed in FIG. 2. In the generic process, a Clock 405 generates a periodic “Clock Signal Every Δt” 410 that is used to synchronize sensor data acquisition and to label time stamps (though other sampling paradigms are conceivable, including variable sampling intervals as might be the case in which data consist of time-stamped occurrences of events such as individual steps or heartbeats).

The processes “Acquire Time-Stamped Data from Physiologic Sensors” 230, “Acquire Time-Stamped Data From Biomechanical Sensors” 235, and “Acquire Time-Stamped Data from Geophysical Sensors” 240 operate simultaneously, in parallel, and according to the same data acquisition scheme; they differ essentially only in the sensors from which they acquire data. In the case of physiologic data sensors, sensed variables include, but are not limited to, heart rate, blood pressure, respiratory rate, and arterial oxygenation saturation. In the case of biomechanical data sensors, sensed variables may include cadence (as in the stride rate of a runner, or the pedaling rate of a cyclist), running stride length, and many other possible measurable parameters related to body movement during exercise. In the case of geophysical data sensors, sensed variables may include GPS coordinates (latitude, longitude, altitude), three dimensions of velocity, and three dimensions of acceleration; variables conveying information related to temperature, barometric pressure, wind speed and direction, and terrain type; as well as other variables.

As indicated by the data acquisition modules, “Acquire Data from Sensor 1” 415, “Acquire Data from Sensor 2” 420, and “Acquire Data from Sensor N” 425, data are acquired from sets of multiple sensors, simultaneously and in parallel at each time point denoted by the Clock Signal A 410. The number of sensors can range from 1 to N, an arbitrarily large number (the ellipsis 423 stands for parallel sensor and data storage modules identical to those numbered 1, 2, and N). As indicated by the data storage modules, “Store Time-Stamped Data: Sensor 1” 430, “Store Time-Stamped Data: Sensor 2” 435, and “Store Time-Stamped Data: Sensor N” 440, all data are stored, once acquired from their associated sensors, in corresponding databases. The stored data entries consist, minimally, of the values of the sensed variables and their corresponding times of acquisition (time stamps).

FIG. 5 diagrams in detail the Calibration sub-mode of the Feedforward mode of the cadence optimization system, the process labeled “Compute Optimized Biomechanical Cadence” 245 in FIG. 2. This sub-process begins by aggregating all acquired sensor data, 505, 510, and 515, into a multidimensional time series stored in a single database 525. The aggregated sensor data is then processed to estimate a cadence optimized for the optimization objective 535 (also identified in process 220 of FIG. 2).

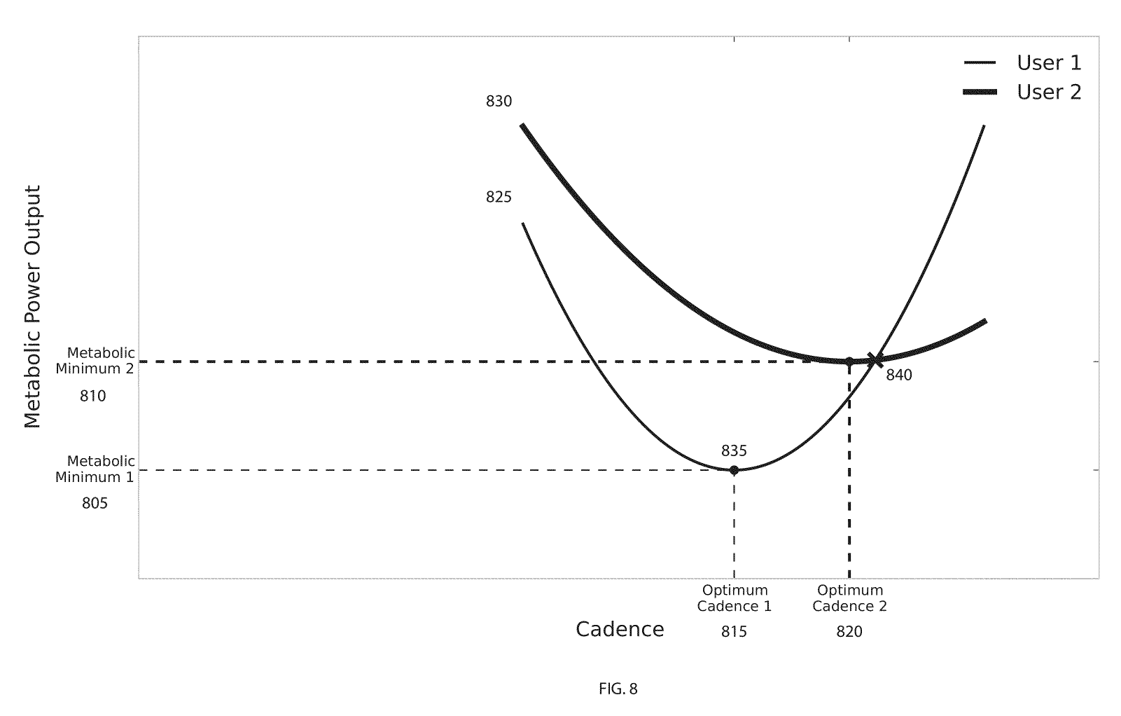

The Estimation Routine to Compute Optimum Cadence from Sensor Data 530 is assisted by observations documented in the physiology literature (Cavanagh, P. R. and K. R. Williams, The Effect of Stride Length Variation on Oxygen Uptake During Distance Running, Medicine and Science in Sports and Exercise, Vol. 14(1), pp. 30-35 (1982)) indicating that metabolic output exhibits second-order functional dependence on cadence when auxiliary parameters such as speed are held constant. The system described here does not depend on such an empirical relationship, and is in principle indifferent to the functional relationships among cadence and other physical and physiologic parameters of interest. In practice, however, the search for optimum cadence is greatly simplified when the functional form of the relationship between cadence and the variable to be optimized is well modeled by a second-order polynomial; the problem that results is then a well posed convex optimization problem, amenable to solution by gradient descent or other techniques well known to those skilled in the art. The Estimation Routine to Compute Optimum Cadence from Sensor Data 530 is explained in more detail with reference to FIG. 8.

During calibration mode, the system will attempt to solve the optimization problem using data generated spontaneously by a user. Existing physiologic literature suggests that even well trained individuals (such as trained athletes) performing specific tasks exhibit natural variation in cadence on the order of several percent. (Cavanagh, P. R. and K. R. Williams, The Effect of Stride Length Variation on Oxygen Uptake During Distance Running, Medicine and Science in Sports and Exercise, Vol. 14(1), pp. 30-35 (1982)). In many practical cases, this natural variation will suffice to permit estimation of the functional dependence of physiologic and other variables of interest on cadence. In all cases, estimation methods known to those skilled in the art are capable of returning, together with estimates of optima, measures of the width of specific confidence intervals around such estimates. In this paradigm, calibration amounts to determining when the quality and amount of data are sufficient to reduce the confidence interval width around the optimum cadence to below that of a predefined threshold 540. In the event that the confidence interval width is not sufficiently narrow 545, the system can prompt the user to generate additional data at unsampled cadences. In particular, gradient descent and other techniques applicable to convex optimization problems can be used to indicate whether higher or lower cadences ought to be sampled 565. The system can then generate an output (feedback error) signal 570 prompting the user to generate data at the required cadences.

As a concrete example, the system may generate music at a higher tempo than the cadences at which a runner has been running in order to prompt the runner to sample faster cadences and enable the system to observe his or her physiologic responses to running at higher cadences, thereby improving the confidence with which the system is able to reconstruct functional relationships between cadence and physiologic parameters of interest). In this way, calibration mode can be understood as a special case of the traditional Feedforward mode, where the objective is to achieve a threshold level of confidence in the estimate of a particular optimum value. In such cases, the output of the Estimation Routine to Compute Optimum Cadence from Sensor Data is a Cadence Value 575 that the system requires to be sampled. This value 575 is then passed to the Music Selection subsystem diagrammed in FIG. 7, which will presently be described.

The system then continues to acquire data through channels 505, 510, and 515, and the Estimation Routine is run iteratively until the confidence threshold 540 is met.

Once the system achieves the desired level of confidence around the optimum cadence 550, the system is capable of returning a cadence value considered optimal 560. This value is stored for future reference as an optimum associated with the specified optimization objective in a user-specific database 120. The optimum cadence 575 is then passed to the Music Selection subsystem diagrammed in FIG. 7, which will presently be described.

FIG. 6 diagrams in detail the Operation mode of the cadence optimization system, also corresponding to the process labeled “Compute Optimized Biomechanical Cadence” 245 in FIG. 2. This process begins by taking as input two selections described earlier in the context of FIG. 2: “Select Mode: Feedback/Feedforward” 210 and 605, and “Select Optimization Objective” 220 and 610. As described in the context of FIG. 5, in Feedforward mode, an optimum cadence associated with each Optimization Objective has been stored following Calibration mode. That optimum cadence is retrieved from memory in the process labeled “Acquire Optimum Cadence” 615. In Operation mode, cadence data is continuously acquired from sensor data, as described in the context of FIG. 4. In the process labeled “Compare Optimum Cadence to Observed Cadence” 625, the observed and optimum cadences are compared 630. If the absolute value of their difference falls below a specified threshold A 640, the system simply outputs the optimum cadence 645. (Note that in Feedback mode, the optimum cadence is defined as equal to the observed cadence, which is continuously acquired from the sensor data 620.)

If the absolute value of the difference between the observed and optimum cadences exceeds the specified threshold Δ 635, the system outputs the optimum cadence together with a feedback (error) signal 650, prompting the user to change cadence to the optimum cadence, or in the direction of the optimum cadence. (In some instantiations, the feedback signal may be transmitted to the user by distorting the audio signal of a musical selection associated with the optimum cadence in ways that emphasize the musical tempo, such as by increasing the amplitude of the bass component.)

FIG. 7 diagrams in detail the subsystem implementing the processes labeled “Select Music with Tempo Matched to Optimized Cadence” 250 and “Play Musical Selection for User” 255. This subsystem contains a Database of Music Selections 710, in which a number of musical selections of uniform tempo have been indexed according to various characteristics, including tempo. The construction of such musical libraries is a common practice, and the technical details of their construction are well known to those skilled in the art. The music selection subsystem takes as input a Cadence Value 705 generated by the Cadence Optimization subsystem described in association with FIG. 5 and FIG. 6. This value is then used to identify musical selections in the database 710 with corresponding tempo. In the event that multiple entries in the database have the indicated tempo, a selection can be made according to any of various rules for disambiguation 715, including but not limited to random selection or prespecified order of preference; such rules may be selected in advance by a user. Audio from chosen musical selection 720 is then played for the user 725.

FIG. 8 illustrates the essential features of the Estimation Routine to Compute Optimum Cadence from Sensor Data, identified as component 530 in FIG. 5. FIG. 8 shows two curves representing the empirically determined functional relationships between metabolic power output (vertical axis) and cadence (horizontal axis) for two hypothetical users running at a fixed speed, modeled after real data presented in Cavanagh, P. R. and K. R. Williams, The Effect of Stride Length Variation on Oxygen Uptake During Distance Running, Medicine and Science in Sports and Exercise, Vol. 14(1), pp. 30-35 (1982). In this example, User 1 (thin line) achieves his minimum metabolic power expenditure at the cadence value labeled “Optimum Cadence 1,” while User 2 (thick line) achieves her minimum metabolic power expenditure at a higher cadence value, labeled “Optimum Cadence 2.” Note that while User 1 is capable of running at the given speed with lower metabolic power output than User 2, there is a cadence (denoted by the ‘X’) above which User 2 is metabolically more efficient.

In particular, FIG. 8 diagrams an example of the different functional relationships between metabolic power output and cadence that might be obtained after the system completes Calibration Mode on two different users, User 1 (thin line 825) and User 2 (thick line 830), subsets of whose data are now considered with respect to running at the same fixed speed. This data is hypothetical but modeled after real data presented in Cavanagh, P. R. and K. R. Williams, The Effect of Stride Length Variation on Oxygen Uptake During Distance Running, Medicine and Science in Sports and Exercise, Vol. 14(1), pp. 30-35 (1982). The smooth curves represent functions derived through curve-fitting (performed using any of a variety of techniques well known to those skilled in the art) from data collected from users observed while walking, running, cycling, or while engaged in another repetitive process for which Metabolic Power Output (plotted as the dependent variable on the vertical axis in FIG. 8) can be studied as a function of repetition frequency (Cadence, plotted as the independent variable on the horizontal axis). The observations used to construct these curves need not be made consecutively; the functions may be estimated on the basis of multiple observations taken at disparate time points. If the users depicted here desire to maximize their endurance at this given running speed, then running at a cadence that minimizes the metabolic power output can be considered optimal. In this example, User 1 achieves his optimum cadence at a lower tempo “Optimum Cadence 1” 815, than does User 2 (“Optimum Cadence 2” 820). Moreover, at their respective optimum cadences we can predict that User 1 will exhibit greater endurance than User 2, since his optimal metabolic power output (“Metabolic Minimum 1” 805) is lower than the optimal metabolic power output of User 2 (“Metabolic Minimum 2” 810). However, this statement is not universally true, since at cadences above the crossing point 840, User 1 will have greater metabolic power output than User 2 and thus have worse endurance; in order to ensure victory, is thus essential that User 1 select the appropriate cadence. This simple yet realistic example demonstrates how the system described in this application can assist users in achieving optimal individual and relative performance.

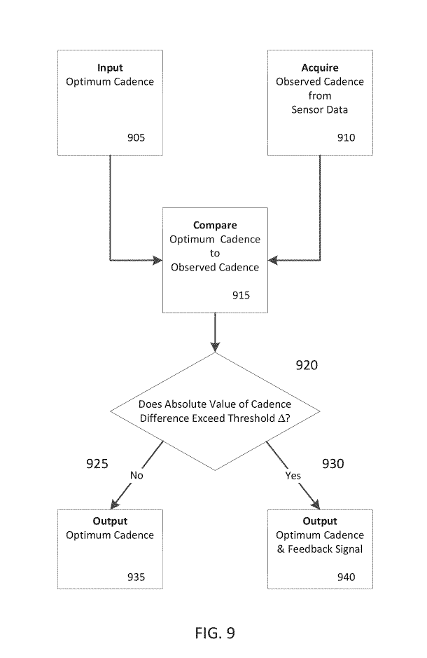

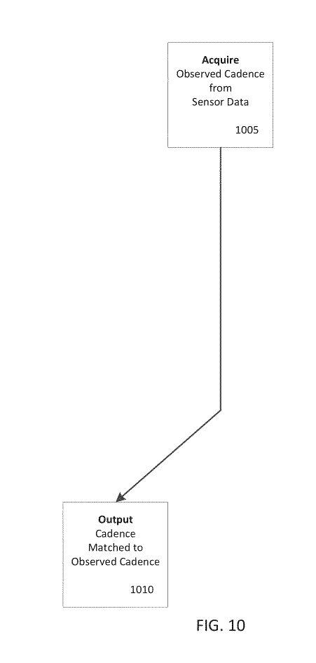

FIG. 9 and FIG. 10 respectively diagram the operation of the system in Feedforward (“Leading”) and Feedback (“Following”) modes, and are both adaptations of FIG. 6, with the modes of operation separated for clarity of exposition.

In Feedforward mode, illustrated in FIG. 9, an Input Optimum Cadence 905 and an Observed Cadence 910 acquired from sensor data are compared (Compare Optimum Cadence to Observed Cadence 915). The Absolute Value of the Cadence Difference obtained in 915 is then evaluated with respect to a tunable threshold, A, and comparison of the absolute difference with A, 920, is then used as a branch point. If the absolute difference is less than or equal to A, as in branch 925, the system Output is Optimum Cadence 935; in this case, the observed cadence is sufficiently close to the optimum cadence that no modification of the observed cadence is considered necessary. If the absolute difference is greater than A, as in branch 930, the system Output is Optimum Cadence and Feedback Signal 940. In this case, the observed cadence is sufficiently far from the optimum cadence that the user receives a signal comprising both the optimum cadence and a supplemental Feedback Signal that is designed to assist the user in modifying activity so as to operate closer to the optimum cadence. The Feedback Signal may indicate the sign of the difference between observed and optimum cadence (whether the operating frequency is faster or slower than optimal), and it may include a supplemental auditory cue, a distortion of the musical selections played for the user (in which the distortion is functionally related to the difference between observed and optimal frequencies). The Feedback Signal may also be constructed in alternative ways.

In Feedback mode, illustrated in FIG. 10, an input cadence designated by the user is Acquired as an Observed Cadence from Sensor Data 1005. FIG. 10 is constructed so as to mirror the format of FIG. 9, demonstrating the relationship between the two modes of operation. The diagram blocks 1010 and 1015 in FIG. 10 are left blank because Feedback mode has no role for processes analogous to the comparison operations 915 and 920, used in Feedforward mode. Instead, in Feedback mode the cadence obtained in 1005 is used directly as the Output Cadence, Matched to Observed Cadence 1020, and is used to drive selections of music with tempo matching the cadence acquired from sensor data in process 1005.

The subject matter described herein can be implemented in digital electronic circuitry, or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (e.g., in a machine readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processor of any kind of digital computer. Generally, a processor will receive instructions and data from a read only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. Information carriers suitable for embodying computer program instructions and data include all forms of nonvolatile memory, including by way of example semiconductor memory devices, (e.g., EPROM, EEPROM, and flash memory devices); magnetic disks, (e.g., internal hard disks or removable disks); magneto optical disks; and optical disks (e.g., CD and DVD disks). The processor and the memory can be supplemented by, or incorporated in, special purpose logic circuitry.

To provide for interaction with a user, the subject matter described herein can be implemented on a computer having a display device, e.g., a CRT (cathode ray tube) or LCD (liquid crystal display) monitor, for displaying information to the user and a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back end component (e.g., a data server), a middleware component (e.g., an application server), or a front end component (e.g., a client computer having a graphical user interface or a web browser through which a user can interact with an implementation of the subject matter described herein), or any combination of such back end, middleware, and front end components. The components of the system can be interconnected by any form or medium of digital data communication, e.g., a communication network. Examples of communication networks include a local area network (“LAN”) and a wide area network (“WAN”), e.g., the Internet.

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods, and systems for carrying out the several purposes of the disclosed subject matter. It is important, therefore, that the claims be regarded as including such equivalent constructions insofar as they do not depart from the spirit and scope of the disclosed subject matter.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter, which is limited only by the claims which follow.

1. A method for determining an optimal repetition rate for a repetitive biomechanical or physiologic process of a user, the method comprising:
defining an optimization objective, wherein the optimization objective comprises minimizing, maximizing, or achieving a specific value of a metabolic cost function;
storing the optimization objective in memory;
monitoring, using a sensor, an actual repetition rate of the biomechanical or physiologic process at a point in time corresponding to the user engaging in the repetitive biomechanical or physiologic process;
monitoring, using the sensor, an actual metabolic cost function value of the biomechanical or physiologic process corresponding to the point in time the actual repetition rate is monitored;
estimating a functional dependence of the actual metabolic cost function value on the actual repetition rate; and
identifying, sing the functional dependence, an optimum repetition rate for the repetitive biomechanical or physiological process with respect to the optimization objective.

2. The method of claim 1, wherein minimizing, maximizing, or achieving a specific value of a metabolic cost function comprises minimizing, maximizing, or achieving a specific value of expended energy during the repetitive biomechanical or physiologic process.

3. The method of claim 1, wherein minimizing, maximizing, or achieving a specific value of a metabolic cost function comprises minimizing, maximizing, or achieving a specific value of fat or carbohydrate metabolism during the repetitive biomechanical or physiologic process.

4. The method of claim 3 comprising constraining the user to walking, running, cycling, dancing, or performing a form of repetitive manual labor within a range of speeds.

5. The method of claim 1, wherein the metabolic cost function identifies the repetition rate associated with the maximum speed at which a user is able to walk, run, cycle, dance, or perform a form of repetitive manual labor.

6. The method of claim 1 comprising constraining the user to cover a particular distance, or constraining the total energy to be expended by the user over a period of time.

7. The method of claim 1, wherein the biomechanical or physiologic process comprises a consciously regulated process comprising one of walking, running, cycling, dancing, or performing a form of repetitive manual labor.

8. The method of claim 1, wherein the biomechanical or physiologic process comprises an unconsciously or autonomically regulated process comprising one of heart rate or respiratory rate.

9. The method of claim 1, wherein the biomechanical or physiologic process comprises a quasiperiodic process.

10. The method of claim 1, wherein the user comprises an animal.

11. The method of claim 1 comprising selecting music matched to the optimal repetition rate.

12. The method of claim 11, wherein the matching of the music to the optimal repetition rate is based on tempo.

13. The method of claim 12, wherein the matching of the music to the optimal repetition rate is based on an observed relationship between specific features of the music and a user's past repetition rate while listening to the music.

14. The method of claim 13, wherein the matching of the music to the optimal repetition rate is based on the observed relationship between specific features of the music and the repetition rates observed in a population of users in performing a repetitive process while listening to said music or music with similar features.

15. The methods of claim 14, wherein the specific features of the music comprise tempo, key, musical genre, artist, or mood.

16. The method of claim 13 comprising playing the selected music for the user to provide synchrony with musical rhythms as a guide to optimizing performance in the repetitive biomechanical or physiologic process.

17. The method of claim 13 comprising distorting the musical selection based on a function of the degree to which the observed repetition rate differs from the rate identified as optimal in order to guide a user in modifying actions to operate at the optimal repetition rate.

18. The method of claim 13, wherein the optimization objective is defined on the basis of an individual achieving a designated metabolic state during rhythmic physical exercise, and wherein music having the appropriate tempo is played for the user as an entrainment signal, to assist the user in moving with an identified optimal repetition rate.

19. A method of matching musical rhythms to physical, biomechanical, and physiologic rhythms of a user, the method comprising:
sensing, using a sensor, an actual repetition rate of a physical, biomechanical, or physiologic process of interest;
automatically selecting music matched by tempo to the actual repetition rate of the process of interest; and
playing the selected music for the user, as the user moves with the detected repetition rate.

20. The method of claim 19 comprising adjusting the selected music as the repetition rate varies over time.

21. The method of claim 19, wherein the biomechanical or physiologic process comprises a consciously regulated process.

22. The method of claim 21, wherein the biomechanical or physiologic process comprises one of walking, running, cycling, dancing, or performing a form of repetitive manual labor.

23. The method of claim 19, wherein the biomechanical or physiologic process comprises one of an unconsciously or autonomically regulated process.

24. The method of claim 23, wherein the unconsciously or autonomically regulated process comprises one of heart rate or respiratory rate.

25. The method of claim 19, wherein the physical process comprises a repetitive process, identified as desirable by a user for the purposes of guiding or automatically matching musical tempo.

26. The method of claim 19, wherein the biomechanical or physiologic process comprises a quasiperiodic process.

27. The method of claim 19, wherein the user comprises an animal.

28. The method of claim 19 comprising aggregating data on a difference between optimal and observed repetition rate for a given musical selection to define a metric describing the musical selection.

29. The method of claim 28, wherein the metric describes the degree to which the observed repetition rate matches the optimal repetition rate while the music is being played.

30. The method of claim 28, wherein the metric is used to guide the music selection.
